(19) United States
(12) Patent Application Publication
(10) Pub. No.: US 2010/0069345 A1
(43) Pub. Date: Mar. 18, 2010

(54) Lanthanide-porphyrin complexes as anti-tumor agents

(76) Inventors: Chi-Ming Che, Hong Kong (HK); Suk-Yu Wong, Hong Kong (HK)

Correspondence Address: COOPER & DUNHAM, LLP, 30 Rockefeller Plaza, 20th Floor, NEW YORK, NY, 10112, US

(21) Appl. No.: 11/582,062

(22) Filed: Oct. 16, 2006

Related U.S. Application Data
(60) Provisional application No. 60/727,866, filed on Oct. 19, 2005.

(51) Int. Cl. A61K 31/555; A61P 35/00

(52) U.S. Cl. 514/184

(57) ABSTRACT

The present invention relates to lanthanide porphyrin complexes and their uses as anti-tumor agents. The described complexes show promising cytotoxic properties toward cancer cells in both in vitro and in vivo studies.

### CROSS-REFERENCE TO RELATED APPLICATION

This application claims the benefit of U.S. Provisional Application No. 60/727,866, filed Oct. 19, 2005, the entire contents of which are incorporated by reference.

### FIELD OF THE INVENTION

The invention is directed to the methods of treating cancer involving administering lanthanide porphyrin complexes as anti-tumor agents.

### BACKGROUND OF THE INVENTION

Cancer refers to a disease state characterized by uncontrolled cell proliferation and differentiation. Cancer cells proliferate excessively and form a local tumor that can potentially invade adjacent normal structures (Bertram G. Katzung, Basic and Clinical Pharmacology, Appleton & Lange, Stanford, Conn., 888). Among many therapeutic options to treat cancer, inorganic medicines are being explored as anti-cancer therapeutics. Cisplatin (cis-diamminedichloroplatinum[II]), discovered by Rosenberg et. al., is one of the most successful anti-cancer drugs known today (B. Rosenberg, L. VanCamp, J. E. Troskp, V. H. Mansour, Nature 1969, 222, 385-386).

Although platinum-based compounds are effective as anti-cancer agents, they exhibit inherent toxicity of heavy metals. Side effects of peripheral sensory neuropathies, hypomaganesemia, nausea and vomiting are commonly found in patients undergoing cisplatin treatment (David S. Fisher, The Cancer Chemotherapy Handbook, St. Louis: Mosby (1997), 78).

With the success of cisplatin, the development of other platinum-based anti-tumor agents has attracted immense interest in recent years. As a consequence, a variety of cisplatin analogs has been synthesized for anti-tumor application (Y.-P. Ho, S. C. F. Au-Yeung, K. K. W. To, Med. Res. Rev. 2003, 2 3, 663-655). Furthermore, there is growing interest to explore the potential use of other metals in anti-cancer drug development (Z. Guo, P. J. Sadler, Angew. Chem. Int. Ed. 1999, 38, 1512-1531) (C.-M. Che, R. W.-Y. Sun, W.-Y. Yu, C.-B. Ko, N. Zhu, H. Sun, J. Chem. Soc., Chem. Commun. 2003, 14, 1718-1719).

Lanthanides, also termed as rare-earth elements or f-block metals, constitute 15 elements with atomic numbers from 57 to 71 (La to Lu). They form trivalent cations with ionic radii similar to, or not much different than that of Ca. The ionic radii of lanthanide(III) ions range from 0.0848 nm (Lu) to 0.1034 nm (Ce) in which the ionic radius of Ca(II) ion is 0.104 nm (M. A. Jakupec, P. Unfried, B. K. Keppler, Rev. Physiol. Biochem. Pharmacol. 2005, 153, 101-111). The cytophysiological effects of lanthanide ions suggest that they may elicit fewer side effects when administered to humans (K. Wang, R. Li, Y. Cheng, B. Zhu, Coord. Chem. Rev. 1999, 190-192, 297-308). Actually, lanthanide metal complexes have found applications as paramagnetic contrast agents with no apparent side-effects (P. Caravan, J. J. Ellison, T. J. McMurry, R. B. Lauffer, Chem. Rev. 1999, 99, 2293-2352). Gadolinium metal complexes, for example, are widely used for the detection of abnormalities of the blood-brain barrier in magnetic resonance imaging (MRI).

However, there are comparatively fewer studies on the use of lanthanide metal complexes as anti-tumor agents. It has been known that lanthanide complexes were used as radiopharmaceuticals and photosensitizers in photodynamic therapy (PDT) (Z. Guo, P. J. Sadler, Angew. Chem. Int. Ed. 1999, 38, 1512-1531). On the other hand, Kostova et. al. reported that lanthanide complexes of bis-coumarins exhibited marginal cytotoxic activities (IC₅₀=9.0×10⁻⁵M to 25.5×10⁻⁶M) against transformed leukemic cell lines (SKW-3, HL-60 and HL-60/Dox) (I. Kostova, I. Manolov, G. Momekov, Eur. J. Med. Chem. 2004, 39, 765-775). Lanthanide complexes of an L-valine derivative of 1,10-phenanthroline also demonstrated significant cytotoxic activities (IC₅₀=6.6×10⁻⁶ M to 12.1×10⁻⁶ M) against a panel of cancer cell lines including HL-60, HCT-8, BGC823, Bel-7402 and KB (Z.-M. Wang, H.-K. Lin, S.-R. Zhu, T.-F. Liu, Y.-T. Chen, J. Inorg. Biochem. 2002, 89, 97-106). These studies highlighted the potential of developing novel lanthanide complexes as anti-cancer therapeutics. However, more analogs would have to be synthesized so as to obtain better activity profiles for this class of compounds.

Cationic porphyrins are known to bind to and stabilize different types of G-quadruplexes and thus inhibit human telomerase (E. Izbicka, R. T. Wheelhouse, E. Raymond, K. K. Davidson, R. A. Lawrence, D. Sun, B. E. Windle L. H. Hurley, D. D. V. Hoff, Cancer Res. 1999, 59, 639-644). Porphyrin compounds as telomerase inhibitors have been patented by Wheelhouse and Hurley in 1998 (R. T. Wheelhouse, and L. H. Hurley WO9833503 1998). Their invention identified compounds with extended aromatic chromophores that bind the G-quadruplex formed by the folding of single-stranded human telomeric DNA. Their invention includes porphyrin compounds and analogs that inhibit telomerase. These compounds include pyridyl and quinolyl porphyrins, with and without a coordinated metal ion, as telomerase inhibitors. Wheelhouse and Hurley claimed the method of modifying the function of telomerase, comprising the interaction of a cationic porphyrin with telomeric DNA.

### SUMMARY OF THE INVENTION

The present invention relates to a method of use of lanthanide porphyrin complexes as anti-tumor agents.

In one embodiment, the invention relates to a method for cancer treatment resulting in the programmed cell death of cancer cells comprising administering to a patient in need thereof a composition comprising an effective amount of a lanthanide porphyrin complex. The lanthanide porphyrin complexes of the present invention can be represented by structural formulas I, II, III and IV:

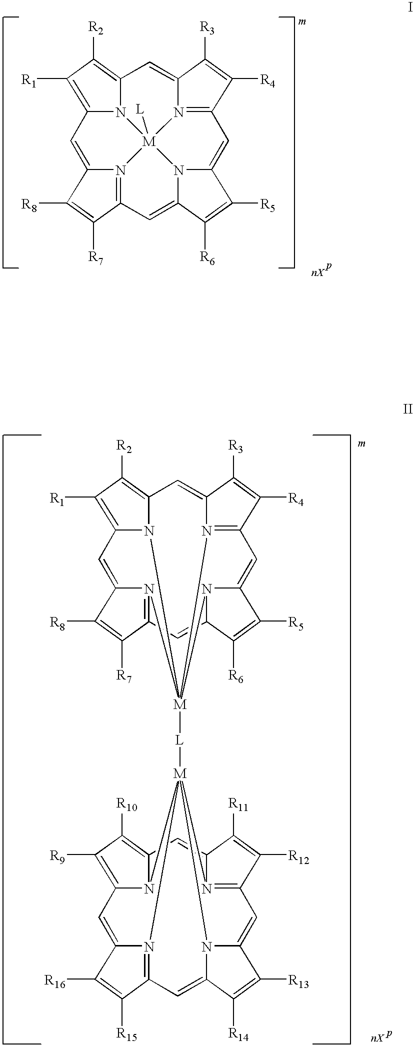

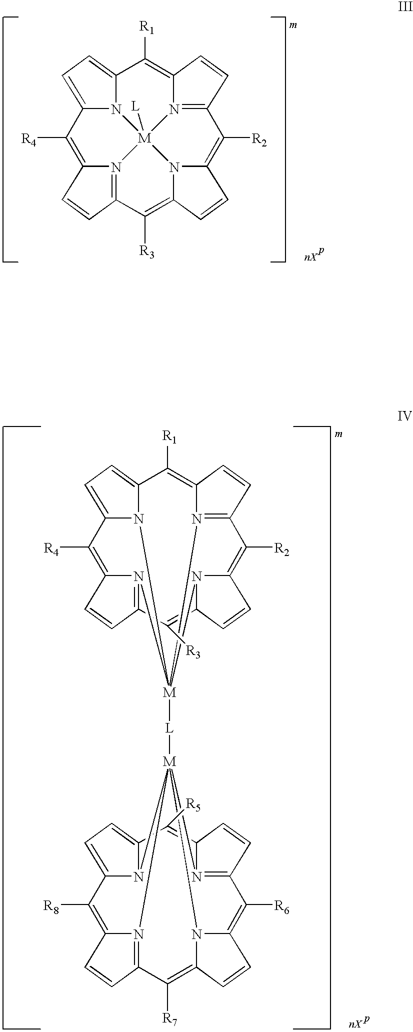

or a pharmaceutically acceptable salt thereof, wherein:

M is a lanthanide metal;

L is any mono-, di-, tri-, tetra- or polydentate ligand; and includes to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s);

each X^{P} is independently a pharmaceutically acceptable counter-ion;
m is an integer ranging from −4 to 1;
P is an integer ranging from −3 to 3;
n is equal to the absolute value of m/p; and

a pharmaceutically acceptable carrier.

In structural formula I and II, R₁-R₈ are each neutral or negatively charged moiety, and are each independently hydrogen, alkyl, substituted alkyl, alkenyl, substituted alkenyl, alkynyl, substituted alkynyl, phenyl, substituted phenyl, halo, nitro, hydroxyl, alkoxyl, substituted alkoxyl, phenoxyl, substituted phenoxyl, aroxyl, substituted aroxyl, alkylthio, substituted alkylthio, phenylthio, substituted phenylthio, phenylthio, substituted phenylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, substituted phosphoramide, C₁-C₂₀ cyclic, substituted C₁-C₂₀ cyclic, heterocyclic, substituted heterocyclic, amino acid, peptide, or polypeptide group; In structural formulas III and IV, wherein R₁-R₄ are each a neutral or negatively charged moiety, and are each independently phenyl, halo substituted phenyl, alkyl substituted phenyl, alkenyl substituted phenyl, alkynyl substituted phenyl, aryl substituted phenyl, cyano substituted phenyl, isocyano substituted phenyl, sulfinyl substituted phenyl, sulfonyl substituted phenyl, sulfonic acid phosphonato substituted phenyl, phosphoramide substituted phenyl, polyaryl substituted phenyl, C₁-C₂₀ cyclic substituted phenyl, amino acid substituted phenyl, peptide substituted phenyl or polypeptide substituted phenyl.

In a preferred embodiment, structural formulas I, II, III and IV is a complex containing a lanthanide ion such as cerium, neodymium, samarium, europium, gadolinium, terbium, dysprosium, holmium, ytterbium and lutetium.

### BRIEF DESCRIPTION OF DRAWINGS

FIG. 1 shows the ORTEP drawing of complex 1 [Yb₂(OEP)₂](μ-OH)₂ with atom-numbering scheme. Hydrogen atoms and solvent molecules are omitted for clarity. Thermal ellipsoids are drawn at the 30% probability level.

FIG. 2 shows the ORTEP drawing of complex 2b [Yb₂(TPP)₂]μ-C₂O₄ with atom-numbering scheme. Hydrogen atoms are omitted for clarity. Thermal ellipsoids are drawn at the 30% probability level.

FIG. 3 shows the UV-visible spectra in dichloromethane of complex 1 at different concentrations.

FIG. 4 shows the UV-visible spectra in dichloromethane of complex 2b at different concentrations.

FIG. 5 shows the determination of the stability of complex 1 (3 μM) in Tris buffer/DMSO (9:1) at different time based on UV-visible analysis: (a) time=0 h, (b) time=2 h and (c) time=24 h.

FIG. 6 shows the determination of the stability of complex 2b (7 μM) in Tris buffer/DMSO (9:1) at different time based on UV-visible analysis: (a) time=0 h, (b) time=2 h and (c) time=24 h.

FIG. 7 shows the determination of the stability of complex 1 (3 μM) in 6 mM GSH Tris buffer/DMSO (9:1) at different time based on UV-visible analysis: (a) time=0 h, (b) time=2 h and (c) time=24 h.

FIG. 8 shows the determination of the stability of complex 2b (7 μM) in 6 mM GSH Tris buffer/DMSO (9:1) at different time based on UV-visible analysis: (a) time=0 h, (b) time=2 h and (c) time=24 h.

FIG. 9 shows the UV-visible spectra of complex 1 (9 μM) in Tris buffer solution with increasing ratio of [DNA]/[1]: 0.01, 0.04, 0.11 and 0.20 at 20° C. Insert: plots of [DNA]/Δεₐₚ against [DNA]. Absorbance was monitored at 410 nm.

FIG. 10 shows the UV-visible spectra of complex 2b (9 μM) in Tris buffer solution with increasing ratio of [DNA]/[1]: 0.01, 0.04, 0.17 and 0.52 at 20° C. Insert: plots of [DNA]/Δεₐₚ against [DNA]. Absorbance was monitored at 430 nm.

FIG. 11 shows the drug sensitivity profiles of (a) human nasopharyngeal carcinoma (SUNE1), (b) its cisplatin-resistant variant (CNE2) toward complexes 1 and 2b and (c) normal human lung fibroblast cell (CCD-19Lu) toward complexes 1, 2a and 2b. Graphs show the percentage of growth compared to the control upon incubation of increasing amounts of lanthanide porphyrin complexes. Drug sensitivity profiles for cisplatin are shown in each graph for comparison.

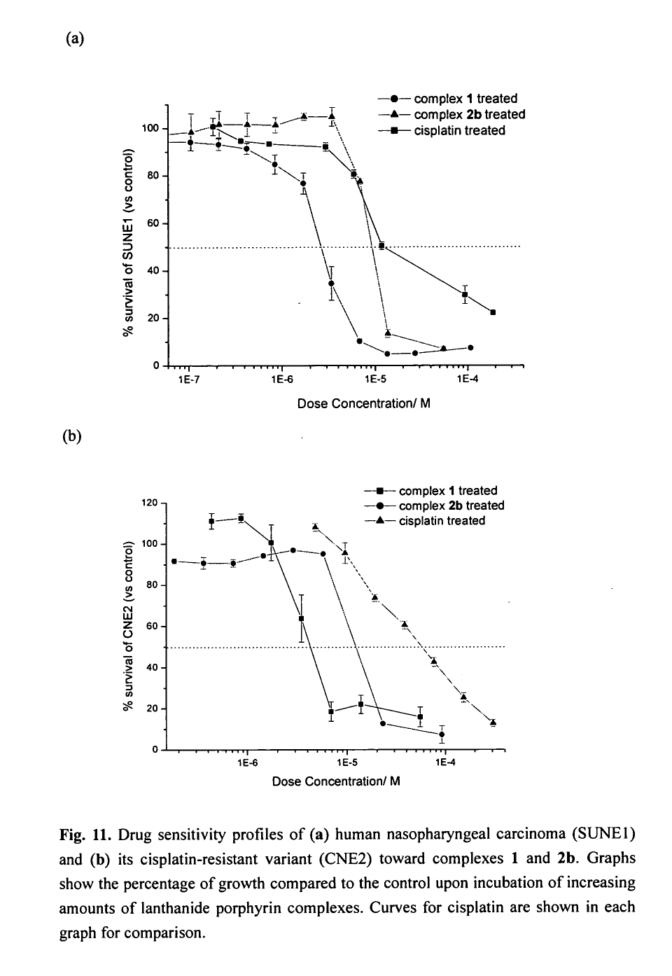

FIG. 12 shows the human cervical epitheloid carcinoma (HeLa) treated with complex 2b (10 μM) for 16 h at 37° C. with 5% CO₂ and were stained by AO/EB and observed under laser scanning confocal microscopy. The apoptotic bodies were marked by circle.

FIG. 13 shows the Cell Cycle analysis of human cervical epitheloid carcinoma (HeLa) with PI staining using Fluorescence-Activated Cell Sorting (FACS). Cells were treated with at 2 μM of complex 1 for T=0 h, T=6 h, T=12 h, T=18 h, T=24 h, T=36 h and T=48 h, respectively. Percentage of cells at G₀-G₁, S, G2-M, and apoptosis at different time intervals were recorded.

FIG. 14 shows the Western blotting analysis of human cervical epitheloid carcinoma (HeLa) treated with complex 1 at 2 μM.

FIG. 15 shows in vivo anti-tumor activity of complex 1 using murine xenograft tumor model.

FIG. 16 shows morphological features of autophagic cells. Transmission electron microscopic photographs of HeLa, (a) untreated control; (b) treated with 1 (1 μM) for 2 h; (c) high magnification of (b); arrows indicate autophagic vacuoles.

FIG. 17 shows the effect of 3-MA on 1-induced cytotoxicity. HeLa cells were pre-treated with 3-MA (10 mM) for 1 h, and then with 1 for 24 h as indicated. Cell viability was measured using MTT assay *, P<0.001.

### DETAILED DESCRIPTION OF THE INVENTION

The present invention is directed to the use of lanthanide porphyrin complexes as anti-tumor agents.

### I. DEFINITIONS

Pharmaceutical composition and the use of lanthanide porphyrin complexes for eradicating cancer cells are provided herein. The pharmaceutical composition contains different synthetic lanthanide porphyrin complexes in an amount effective to induce apoptosis of cancer cells.

It will be understood that the ligand and the cationic metal center may not form a charged neutral complex. For instance, the net positive charge on the cationic metal may be greater than the absolute net negative charge of the deprotonated macrocyclic ligand; or the net positive charge on the cationic metal may be less than the absolute net negative charge of the deprotonated macrocyclic ligand. In view of this, there should be least one anion or counter-ion coordinated to the metal center for charge neutralization. Accordingly, the phrase “pharmaceutically acceptable salt,” as used herein, this includes salts formed from charged metal complex and the anion or counter-ion.

As used herein, the phrase “counter-ion” refers to an ion associated with a positively or negatively charged lanthanide porphyrin complex. Non-limiting examples of counter-ion include fluoride, chloride, bromide, iodide, sulfate, phosphate, trifluoromethanesulfonate, acetate, nitrate, perchlorate, acetylacetonate, hexafluoroacetylacetonate, sodium and potassium.

As used herein, the term “ligand” refers to an ion or molecule that binds to lanthanide complexes of the invention. It includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s).

As used herein, the term “OEP” represents the dianionic octaethylporphyrin with —C₂H₅ substituents at positions 1-8.

As used herein, the term “TPP” refers to tetraphenylporphyrin dianion with phenyl substituents at bridging α,β,γ,δ positions and pyrrole 1-8 positions unsubstituted.

As used herein, the term “GSH” refers to reduced glutathione.

As used herein, the phrase of “lanthanide porphyrin complex” as used herein refers to any metals in the lanthanide series bound to any dianionic synthetic porphyrins. The metal can have one or more various neutral ligands or negatively charged ligands. The structure of lanthanide porphyrin complexes can be either in monomeric, dimeric or polymeric form. Also, they can exist as a single molecule or aggregated molecules.

As used herein, the phrase of “pharmaceutically acceptable carrier” means approved by a regulatory agency of the Federal or a state government or listed in the U.S. Pharmacopiea or other generally recognized pharmacopeia for use in animals, mammals, and more particularly in humans. Non-limiting examples of pharmaceutically acceptable carriers include liquids, such as water and oils, including those of petroleum, animal, vegetable, or synthetic origin. Water is preferred vehicle when the compound of the invention is administered intravenously. Saline solutions and aqueous dextrose and glycerol solutions can also be employed as liquid vehicles, particularly for injectable solutions.

As used herein, the term “patient” refers to any member of the kingdom Animalia. Non-limiting examples of animals include a cow, monkey, horse, sheep, pig, cat, dog, mouse, rat, rabbit, and guinea pig and most preferably a human.

As noted above, the present invention relates to compositions useful for the induction of programmed cell death of cancer cells.

In one embodiment, the invention relates to a method for induction of programmed cell death (including but not limited to apoptosis) of cancer cells comprising administering to a patient in need thereof a composition comprising an effective amount of lanthanide porphyrin complexes. The lanthanide porphyrin complexes of this invention can be represented by structural formula I, II, III and IV or a pharmaceutically acceptable salt thereof.

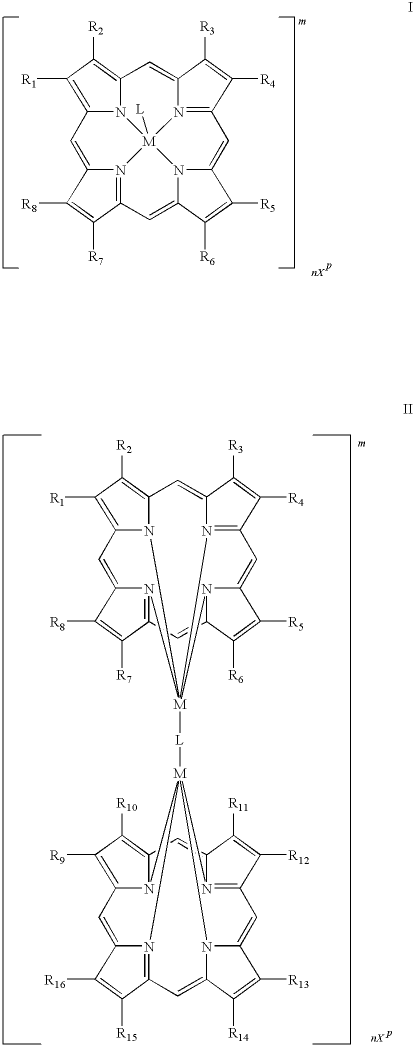

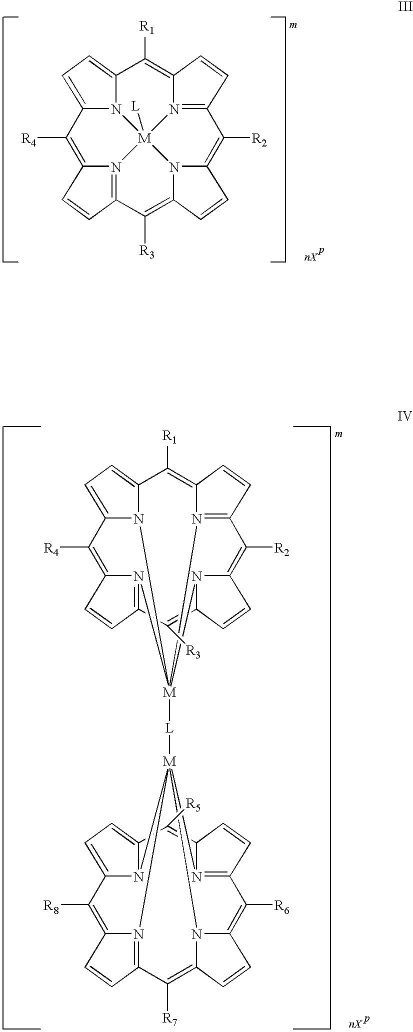

In a preferred embodiment, structural formula I, II, III and IV is a complex containing a metal ion, preferably in the lanthanide series. Representative metals include cerium, neodymium, samarium, europium, gadolinium, terbium, dysprosium, holmium, ytterbium and lutetium.

In another embodiment, L in the structural formula I, II, III and IV is any mono-, di-, tri-, tetra- or polydentate ligand; and L includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s) or oxalate(s).

In structural formula I and II, R₁-R₈ are each neutral or negatively charged moiety, and are each independently hydrogen, alkyl, substituted alkyl, alkenyl, substituted alkenyl, alkynyl, substituted alkynyl, phenyl, substituted phenyl, halo, nitro, hydroxyl, alkoxyl, substituted alkoxyl, phenoxyl, substituted phenoxyl, aroxyl, substituted aroxyl, alkylthio, substituted alkylthio, phenylthio, substituted phenylthio, phenylthio, substituted phenylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, substituted phosphoramide, C₁-C₂₀ cyclic, substituted C₁-C₂₀ cyclic, heterocyclic, substituted heterocyclic, amino acid, peptide, or polypeptide group.

In structural formula III and IV, wherein R₁-R₄ are each neutral or negatively charged moiety, and are each independently phenyl, halo substituted phenyl, alkyl substituted phenyl, alkenyl substituted phenyl, alkynyl substituted phenyl, aryl substituted phenyl, cyano substituted phenyl, isocyano substituted phenyl, sulfinyl substituted phenyl, sulfonyl substituted phenyl, sulfonic acid phosphonato substituted phenyl, phosphoramide substituted phenyl, polyaryl substituted phenyl, C₁-C₂₀ cyclic substituted phenyl, amino acid substituted phenyl, peptide substituted phenyl or polypeptide substituted phenyl.

In another embodiment, the invention relates to a method for the induction of programmed cell death comprising administering to a patient in need thereof a composition comprising an effective amount of a lanthanide porphyrin complex of formula II or a pharmaceutically acceptable salt thereof, wherein R₁, R₂, R₃, R₄, R₅, R₆, R₇, R₈, R₉, R₁₀, R₁₁, R₁₂, R₁₃, R₁₄ R₁₅ and R₁₆ are each -ethyl; M is ytterbium; L is two hydroxyls; m is 0; X^{P} is absent and a pharmaceutically acceptable carrier.

In another embodiment, the invention relates to a method for the induction of programmed cell death comprising administering to a patient in need thereof a composition comprising an effective amount of a lanthanide porphyrin complex of formula IV or a pharmaceutically acceptable salt thereof, wherein R₁, R₂, R₃, R₄, R₅, R₆, R₇ and R₈ are each -phenyl; M is ytterbium, L is an oxalate; m is 0; X^{P} is absent and a pharmaceutically acceptable carrier.

In another embodiment, the invention relates to a method for the inhibition of tumor growth in a murine xenograft mice model using the multi-drug resistant QGY-TR50 human liver cancer cell line comprising administering to a patient in need thereof an effective amount of the lanthanide porphyrin complexes of formula I, II, III and IV or a pharmaceutically acceptable salt.

### II. NATURAL AND SYNTHETIC PORPHYRINS

Porphyrins are tetrapyrrole macrocyclic compounds with methylene bridges connecting each pyrrole unit. Porphyrins can be categorized as naturally occurring in origin or synthetic in origin.

Natural porphyrins play a vital role in oxygen-storage and oxygen-transport in biological systems. For instance, the iron porphyrin moiety (the heme unit) is the prosthetic group present in myoglobin and hemoglobin (the oxygen carrier), and various oxygenases (a class of enzymes that incorporate one or two atoms from oxygen to a substrate).

Synthetic porphyrins are those made via condensation of aldehyde and pyrrole or built up from smaller organic fragments. All porphyrins can have substituents at any of the positions of the ring periphery, including the pyrrole positions and the meso (bridging methylene) positions as well as at the central nitrogen atoms. There can be one or more substituents, and combinations of one or more different substituents (K. M. Kadish, K. M. Smith, R. Guilard, The Porphyrin Handbook 1998, Volume I-XV).

The synthesis and chemistry of porphyrins are well-documented (K. M. Kadish, K. M. Smith, R. Guilard, The Porphyrin Handbook 1998, Volume I-XV) (A. D. Adler, J. Org. Chem. 1967, 32, 476-476).

### III. LANTHANIDE COMPLEXES IN BIOMEDICAL APPLICATION

The pharmaceutical use of lanthanide(III) metal complexes as paramagnetic contrast agents for magnetic resonance imaging (MRI) is well established. Gadolinium(III) metal complexes, for example, are widely used for the detection of abnormalities of blood-brain barrier. In addition, their uses as radiopharmaceuticals and photosensitizers in photodynamic therapy (PDT) have intrigued bioinorganic chemists. Lutetium texaphyrin complex is an exemplary example currently on clinical trial as a photosensitizer for the treatment of cancer and atherosclerosis (P. J. Sadler, Z. Guo, Angew. Chem. Int. Ed. 1999, 38, 1512-1531).

### IV. SYNTHESIS OF LANTHANIDE (III) PORPHYRIN COMPLEXES

In general, lanthanide (III) porphyrin complexes can be synthesized by reacting the appropriate lanthanide salt with the desired porphyrin at high temperature under inert gas atmosphere (W.-K. Wong, L. Zhang, W.-T. Wong, F. Xue, T. C. W. Mak J. Chem. Soc., Dalton Trans 1999, 615-622) (W.-K. Wong, L. Zhang, W.-T. Wong, F. Xue, T. C. W. Mak J. Chem. Soc., Dalton Trans 1999, 3053-3062) (T. S. Srivastava, Bioinorg. Chem. 1978, 8, 61-76) (T. S. Srivastava, T. Yonetani Chem. Phys. Lett. 1976, 40, 456-461). After cooling, the reaction mixture was dissolved in 50 ml of CH₂Cl₂ and 5 ml of MeOH. The residual was filtered off and the solution was washed with 50 ml of water twice. The solution was then dried over anhydrous Na₂SO₄. The dried solution was concentrated and then applied to a MgCO₃ column. Unreacted free porphyrin was eluted with CH₂Cl₂:Hexane (2:1). Product of lanthanide porphyrin was eluted with CH₂Cl₂:MeOH (5:1).

In this invention, the compositions described herein are lanthanide porphyrin complexes having the following structure II and IV:

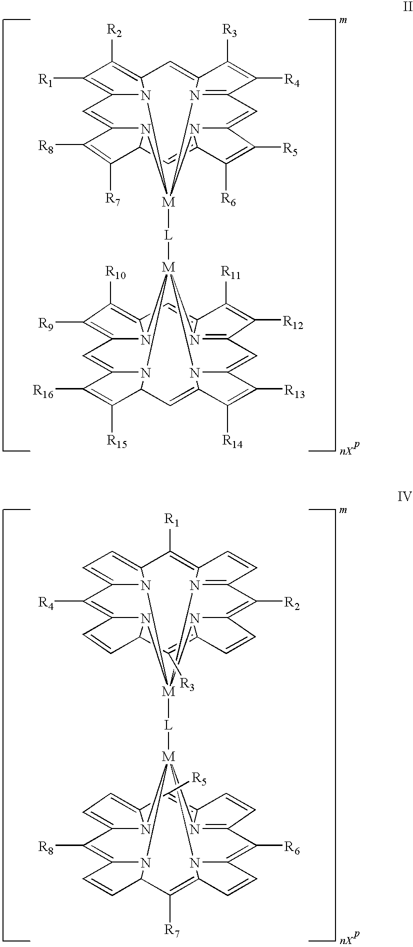

Exemplary synthetic lanthanide (III) porphyrin complexes of formula II and IV are given below.

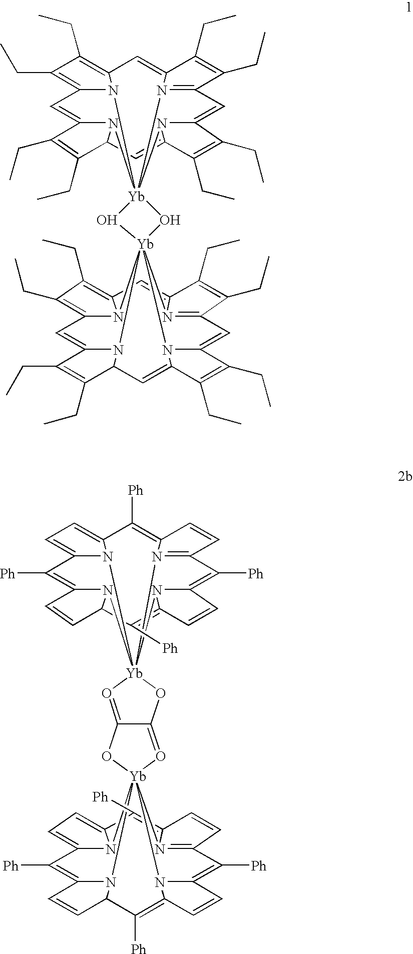

Complexes were synthesized according to the procedure mentioned above. With complex 2b, 1% acetic acid was added to methanol to elute the product. High purity complexes were obtained by successive recrystallization of compounds. The compound(s) is/are characterized by mass spectroscopy (FAB), UV-visible spectroscopy, X-ray crystallography and elemental analyse.

### V. ANTI-CANCER ACTIVITY OF LANTHANIDE PORPHYRIN COMPLEXES

To our knowledge, the lanthanide porphyrin complexes in this invention exhibit more potent activity against human cancer than other lanthanide complexes known in art. The cytotoxic activity of one of the lanthanide complexes toward breast cancer cell line is 30 times higher than that of cisplatin. In addition, the lanthanide porphyrin complexes exhibit similar cytotoxic activities on both the parental human nasopharyngeal carcinoma and a cisplatin-resistant derivative of it. Most importantly, complex 1 exhibits prominent cytotoxic effect toward the human liver cancer cell line (QGY-7703) (IC₅₀=0.06±0.01 μM). Compared with cisplatin, complex 1 is 760 times more effective in eradicating human liver cancer cell line.

Non-limiting examples of cancer cells include mammalian carcinoma cells including, but not limited to human cervical epitheloid carcinoma cells, human breast carcinoma cells, human nasopharyngeal carcinoma cells, human liver carcinoma cells and their multi-drug resistant variants.

In anti-cancer therapeutics, nucleic acids are one of the prime targets for pharmaceutical interventions. By incorporating into elongating DNA or RNA (nucleoside analogs), or binding to DNA by covalent bonds (alkylating agents), or by binding to DNA grooves (intercalating agents), these agents will interfere with cell cycle progression, leading to inhibition in cell growth. Since the porphyrin structure is known to interact with DNA, we tested the interaction between lanthanide (III) porphyrin complexes and DNA. The interaction between the two can be studied using spectroscopic methods such as fluorescence and electronic absorption spectroscopy (C. V. Kumar, E. H. Asuncion, J. Am. Chem. Soc. 1993, 115, 8547).

In this invention, the lanthanide porphyrin complexes were found to have strong interaction with calf thymus DNA in the electronic absorption spectroscopy. The binding constants of lanthanide porphyrin complexes were found to be strong (K=10⁶ mol⁻¹dm³) with moderate hyprochromicity around 27%.

### VII. EXAMPLES

The following examples are set forth to assist in understanding the invention and should not be construed as specifically limiting the invention described and claimed herein.

Materials. All chemicals unless otherwise stated were purchased from Sigma-Aldrich Chemical Co. (Tokyo, Japan). Analytical grade organic solvents and double distilled deionized water were used throughout the experiments. Ytterbium salts was purchased from Strem Chemicals Inc.

The calf thymus DNA was purchased from Sigma-Aldrich Chemical Co. and was purified by phenol-chloroform extraction. The DNA was dissolved in buffer (5 mM Tris, 5 mM NaCl, pH 7.2). The concentration (per base pair) or calf thymus DNA was determined spectophotometrically on the basic of ε₂₆₀, 13200 M⁻¹cm⁻¹bp.

Human cervical epitheloid carcinoma cells (HeLa), human hepatocellular carcinoma cells (HepG2 and Hep3B), human breast cancer cells (MCF-7), human coloadenocarcinoma cells (HCT-8), and normal human lung fibroblast cells (CCD-19Lu) were purchased from American Type Culture Collection (ATCC) Rockville, Md., USA. The human hepatocellular cancinoma cells QGY-7703, and the multi-drug resistant cell line QGY-TR50 derived from QGY-7703, were generously provided by Prof. Yong Xie (The Hong Kong University of Science and Technology, Hong Kong PRC). QGY-TR50 cells were isolated and cultured as described (J. Zhou, S. C. Cheng, D. Luo, Y. Xie Biochem. Biophys. Res. Commun. 2001, 280, 1237-1242). Human nasopharyngeal carcinoma cells (SUNE1 and its cisplatin-resistant variant—CNE2) were derived from poorly differentiated NPC in Chinese patients and were generously provided by Dr. S. W. Tsao (The University of Hong Kong, Hong Kong PRC). Cell culture flasks and 96-well microtitre plates were from Nalge Nunc Int. Culture medium, other medium constituents and phosphate buffer saline (PBS) were obtained by Gibco BRL Rockville, Md., USA. Cell Proliferation kit I (MTT) was purchased from Roche. Mannheim, Germany.

The murine model for anti-tumor evaluation in vivo was the male BALB/c-nu/nu mouse originated from Charles River Laboratories, Inc. and the colony was maintained in the Laboratory Animal Unit, The University of Hong Kong. Mice were grafted in the left flank with QGY-TR50 cells subcutaneously. All of the experiments were carried out according to the guidelines of the Committee on the Use of Live Animals in Teaching and Research, The University of Hong Kong.

Instrumentation. All absorption spectra were recorded on a Perkin-Elmer Lambda 900 and Perkin-Elmer Fusion α-FP. Positive ion FAB mass spectra were recorded on a Finnigan MAT95 mass spectrometer. ¹HNMR spectra were recorded on Brucker DPX-300 or DPX-400 NMR spectrometers. Flow cytometric analysis was performed with a Coulter EPICS flow cytometer (Coulter, Miami, Fla.) equipped with 480 long, 525 band and 625 long pass mirrors. Samples were excited by 15 mW air-cool argon convergent laser at 488 nm. Fluorescence signal were manipulated with Coulter Elite 4.0 software (Coulter) and were analyzed by Winlist 1.04 and Modfit 5011 software (verity Software House, Topsham, Me.).

The identities of the complexes were characterized by X-ray crystallography, FAB-MS, UV-visible spectroscopy, ¹HNMR and further confirmed by microanalysis (elemental analysis).

### Example 1

### Synthesis of Lanthanide Porphyrin Complexes

Example 1 describes the preparation and characterization of illustrative lanthanide porphyrin complexes 1, 2a and 2b.

Lanthanide(III) porphyrin complex 1. The -μ-OH bridging ytterbium(III) porphyrin complex [Yb₂(OEP)₂](μ-OH)₂ was synthesized by reacting the anhydrous lanthanide chloride with the desired porphyrin in imidazole at high temperature under inert gas atmosphere (T. S. Srivastava, Bioinorg. Chem. 1978, 8, 61-76) (T. S. Srivastava, T. Yonetani Chem. Phys. Lett. 1976, 40, 456-461). After cooling, the reaction mixture was dissolved in a mixture of CH₂Cl₂ (50 ml) and MeOH (5 ml). The residual was filtered off and the solution was washed with 50 ml of water twice. The solution was then dried over anhydrous Na₂SO₄. The dried solution was concentrated and then applied to a MgCO₃ column. Unreacted free porphyrin was eluted with CH₂Cl₂:Hexane (2:1). Product of lanthanide porphyrin was eluted with CH₂Cl₂:MeOH (5:1). High purity of compound was obtained by successive recrystallization of compounds in CH₂Cl₂/pentane. Crystals of 1 were obtained in diethyl ether for X-ray crystallography. The compound was then characterized by mass spectroscopy, UV-visible spectroscopy, ¹HNMR and further confirmed by elemental analysis.

Lanthanide(III) porphyrin complex 2a. Complex 2a was synthesized according to the literature with some modifications (C. P. Wong, Inorganic Syntheses 1983, 22, 156-62). After solvent removal, the compound was redissolved in CH₂Cl₂ and the residue was filtered off. It was then chromatographed on a MgCO₃ column. Unreacted free porphyrin was eluted with CH₂Cl₂:Hexane (1:2). Product of lanthanide porphyrin was eluted with CH₂Cl₂ and finally with ethyl acetate. High purity of product was obtained by successive recrystallization of compounds in CH₂Cl₂/pentane. The compound was then characterized by mass spectroscopy, UV-visible spectroscopy and further confirmed by elemental analysis.

Lanthanide(III) porphyrin complex 2b. Compound 2b was synthesized using synthetic procedure of complex 1 with slight modifications. After eluting the unreacted porphyrin with CH₂Cl₂:Hexane (1:2), 1% acetic acid in methanol was applied to the MgCO₃ column to obtain complex 2b. High purity of compound was obtained by successive recrystallization of compounds using CH₂Cl₂/pentane. Crystals of 2b were obtained in ethyl acetate/pentane for X-ray crystallography. The compound was then characterized by UV-visible spectroscopy and further confirmed by elemental analyse.

Analytical data for complexes 1, 2a and 2b was shown below:

Complex 1: Yield: 78%. Anal. Calcd. for C₇₂H₈₈N₈Yb₂O₂.2H₂O (%): C, 58.39; H, 6.22; N, 7.57. Found: C, 58.06; H, 6.42; N, 7.63. UV-visible (CH₂Cl₂) λₘₐₓ/nm (ε/dm³mol⁻¹ cm⁻¹): 404(257000), 535(10500), 572(14800). ¹HNMR (CDCl₃): δ21.3 (s, 4H), δ12.3 (s, 8H), δ9.2 (s, 8H), δ3.7 (s, 24H). FAB MS: m/z 724 [M/2+H]

Complex 2a: Yield: 52%. Anal. Calcd. for C₄₉H₃₅N₄YbO₂.3H₂O (%): C, 64.69; H, 4.51; N, 6.16. Found: C, 64.41; H, 4.60; N, 5.49. UV-visible (CH₂Cl₂) λₘₐₓ/nm (ε/dm³ mol⁻¹ cm⁻¹): 422(603000), 553(19500), 591(4100). FAB MS: m/z 885 [M+H]⁺.

Complex 2b: Yield: 69%. Anal. Calcd. for C₄₆H₃₁N₄YbO₂.CH₃OH (%): C, 64.38; H, 4.00; N, 6.39. Found: C, 64.78; H, 3.79; N, 6.54. UV-visible (CH₂Cl₂) λₘₐₓ/nm (ε/dm³mol⁻¹cm⁻¹): 422(245000), 550(12600), 591(2700).

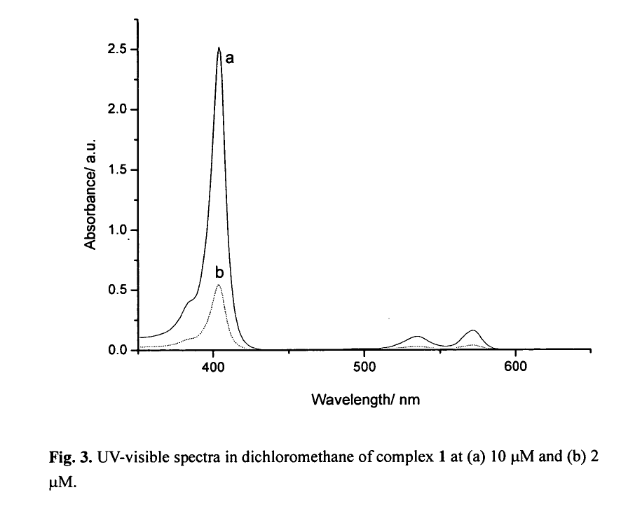

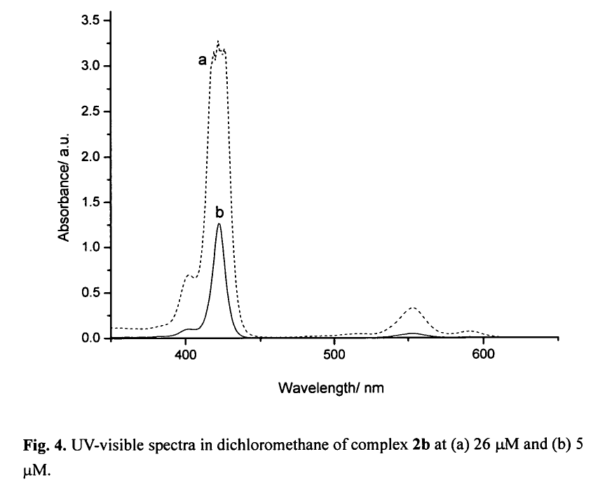

The UV-visible spectra of complexes 1 and 2b were also shown in FIGS. 3 and 4. Both complexes feature an intense Soret band at 404 nm (complex 1) and 422 nm (complex 2b) and two weaker Q bands.

X-ray Crystallography. Crystals of complexes 1 and 2b were obtained and data was recorded as follow:

Complex 1. A red crystal with dimensions 0.3×0.2×0.05 mm³ mounted in a glass capillary was used for data collection at −20° C. on an MAR diffractometer with a 300 mm image plate detector using graphite monochromatized Mo—K_{α} radiation (λ=0.71073 Å). Data collection was made with 2° oscillation step of φ, 900 seconds exposure time and scanner distance at 120 mm. 100 images were collected. The images were interpreted and intensities were integrated using program DENZO (Z. Otwinowski, W. Minor, In Processing of X-ray Diffraction Data Collected in Oscillation Mode, Methods in Enzymology, C. W. Carter, Jr. Sweet, Eds, R. M.; Academic Press: 1997, 276, 307-326). The structure was solved by direct methods employing SHELXS-97 program (G. M. Sheldrick, SHELX97. Programs for Crystal Structure Analysis (Release 97-2), University of Goetingen, Germany, 1997).

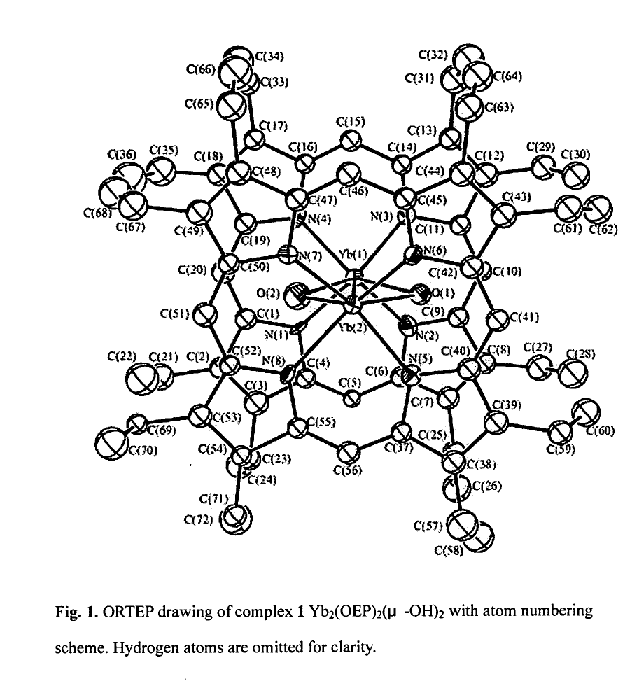

The molecular structure of complex 1 was established by X-ray crystallography (FIG. 1). Crystallographic data collection parameters and selected bond angles and distances for complex 1 were summarized in Table 1a and 1b, respectively. From FIG. 1, the ytterbium(III) ion was in seventh coordination. The metal center sat 1.073 Å above the centroid of the porphyrin plane in which the angle sum between the four pyrrolic nitrogens and the metal center was 315.5°. In order to accommodate the relatively large metal center, the planarity of the porphyrin ring vanished.

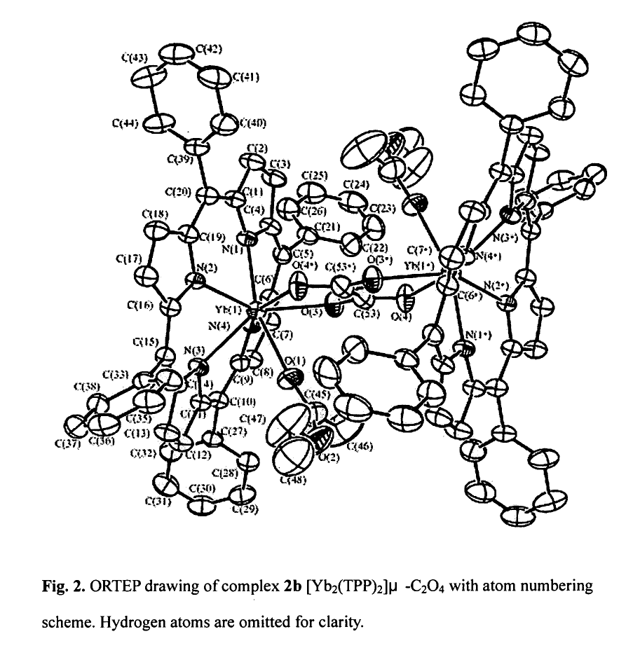

The molecular structure of complex 2b was established by X-ray crystallography (FIG. 2). Crystallographic data collection parameters and selected bond angles and distances for complex 2b were summarized in Table 2a and 2b, respectively. The ytterbium (III) ion was also in seventh coordination with one ethyl acetate ester coordinated (FIG. 2). Two ytterbium (III) ions were bridged by an oxalate and each ytterbium sat slightly above the plane of the porphyrin. The planarity of the porphyrin ring was also found to lose. In views of bond distances, the Yb—N distances in complex 2b are 2.17, 2.26, 2.26 and 2.38 Å, in which they are comparable to the literature values 2.310-2.339 Å (T. J. Foley, K. A. Abboud, J. M. Boncella, Inorg. Chem. 2002, 41, 1704-1706).

TABLE 1a
X-ray crystallographic Data for complex 1.

| Empirical formula | C₇₂H₈₈N₈O₂Yb₂•2H₂O | |
|---|---|---|
| Formula weight | 1479.62 | |
| Temperature | 253(2) K | |
| Wavelength | 0.71073 Å | |
| Crystal system | Monoclinic | |
| Space group | P 2₁/n | |
| Unit cell dimensions | a = 21.626(4) Å | α = 90° |
| | b = 15.247(3) Å | β = 115.48° |
| | c = 23.238(5) Å | γ = 90° |

| | |
|---|---|
| Volume | 6917(2) Å³ |
| Z | 4 |
| Density (calculated) | 1.421 Mg/m³ |
| Absorption coefficient | 2.739 mm⁻¹ |
| F(000) | 3008 |
| Crystal size | 0.3 × 0.2 × 0.05 mm³ |
| Theta range for data collection | 1.08 to 23.78°. |
| Index ranges | −21 <= h <= 20, −15 <= k <= 16, |
| | −25 <= l <= 24 |
| Reflections collected | 22824 |
| Independent reflections | 6895 [R(int) = 0.0945] |
| Completeness to theta = 24.16° | 65.2% |
| Refinement method | Full-matrix least-squares on F² |
| Data/restraints/parameters | 6895/0/407 |
| Goodness-of-fit on F² | 0.855 |
| Final R indices [I > 2sigma(I)] | R1 = 0.064, wR2 = 0.151 |
| R indices (all data) | R1 = 0.152, wR2 = 0.166 |
| Largest diff. peak and hole | 1.268 and −1.386 eÅ⁻³ |

TABLE 1b
Selected Bond Distances (Å) and Bond Angles (deg) for Complex 1.
Bond Distances (Å)

| | |
|---|---|
| Yb (1)—N (2) | 2.17 (3) |
| Yb (1)—N (1) | 2.26 (2) |
| Yb (1)—N (4) | 2.26 (2) |
| Yb (1)—N (3) | 2.38 (2) |

| | |
|---|---|
| Bond Angle (deg) | Bond Angle (deg) |

| | | | | | |
|---|---|---|---|---|---|
| N (2)—Yb (1)—N (1) | 82.3 | (9) | N (2)—Yb (1)—N (4) | 130.7 | (10) |
| N (1)—Yb (1)—N (4) | 75.5 | (9) | N (2)—Yb (1)—O (6) | 85.7 | (10) |
| N (1)—Yb (1)—O (6) | 85.5 | (10) | N (4)—Yb (1)—O (6) | 134.3 | (10) |
| N (2)—Yb (1)—N (3) | 79.6 | (9) | N (1)—Yb (1)—N (3) | 124.8 | (8) |
| N (4)—Yb (1)—N (3) | 78.1 | (9) | N (2)—Yb (1)—O (2) | 150.1 | (9) |
| N (4)—Yb (1)—O (2) | 74.4 | (10) | N (4)—Yb (1)—O (1) | 124.6 | (10) |
| N (3)—Yb (1)—O (2) | 93.0 | (9) | N (2)—Yb (1)—O (1) | 93.2 | (11) |
| N (3)—Yb (1)—O (1) | 79.6 | (8) | O (2)—Yb (1)—O (1) | 56.9 | (11) |

TABLE 2a
X-ray crystallographic Data for complex 2b.

| Empirical formula | C₉₀H₅₆N₈O₄Yb₂•4C₄H₈O₂ | |
|---|---|---|
| Formula weight | 2011.92 | |
| Temperature | 301(2) K | |
| Wavelength | 0.71073 Å | |
| Crystal system | Monoclinic | |
| Space group | P 2₁/n | |
| Unit cell dimensions | a = 13.284(3) Å | α = 90° |
| | b = 15.184(3) Å | β = 99.95° |
| | c = 22.582(5) Å | γ = 90° |

| | |
|---|---|
| Volume | 4486.4(16) Å³ |
| Z | 2 |
| Density (calculated) | 1.489 Mg/m³ |
| Absorption coefficient | 2.141 mm⁻¹ |
| F(000) | 2032 |
| Crystal size | 0.4 × 0.3 × 0.15 mm³ |
| Theta range for data collection | 1.83 to 25.51°. |
| Index ranges | −15 <= h <= 15, −18 <= k <= 18, |
| | −27 <= l <= 27 |
| Reflections collected | 31291 |
| Independent reflections | 7776 [R(int) = 0.0464] |
| Completeness to theta = 25.51° | 93.0% |
| Absorption correction | None |
| Refinement method | Full-matrix least-squares on F² |
| Data/restraints/parameters | 7776/0/577 |
| Goodness-of-fit on F² | 0.942 |
| Final R indices [I > 2sigma(I)] | R1 = 0.0338, wR2 = 0.0830 |
| R indices (all data) | R1 = 0.0468, wR2 = 0.0854 |
| Largest diff. peak and hole | 0.850 and −1.399 eÅ⁻³ |

TABLE 2b
Selected Bond Distances (Å) and Bond Angles (deg) for Complex 2b.

| | |
|---|---|
| Bond Distances (Å) | Bond Distances (Å) |

| | | | |
|---|---|---|---|
| Yb (1)—O (4)#1 | 2.292 (3) | Yb (1)—O (3) | 2.315 (3) |
| Yb (1)—N (2) | 2.309 (3) | Yb (1)—N (1) | 2.315 (3) |
| Yb (1)—N (4) | 2.319 (3) | Yb (1)—N (3) | 2.330 (3) |

| Bond Angle (deg) | | Bond Angle (deg) | |
|---|---|---|---|
| N (4)#1—Yb (1)—N (1) | 77.62 (11) | N (2)—Yb (1)—N (4) | 124.88 (11) |
| N (1)—Yb (1)—N (4) | 77.76 (11) | N (2)—Yb (1)—N (3) | 76.36 (10) |
| N (1)—Yb (1)—N (3) | 123.35 (12) | N (4)—Yb (1)—N (3) | 77.54 (11) |

### Example 2

### Stability Studies of Lanthanide Porphyrin Complexes

Example 2 describes the results of stability studies for illustrative lanthanide porphyrin complexes 1 and 2b.

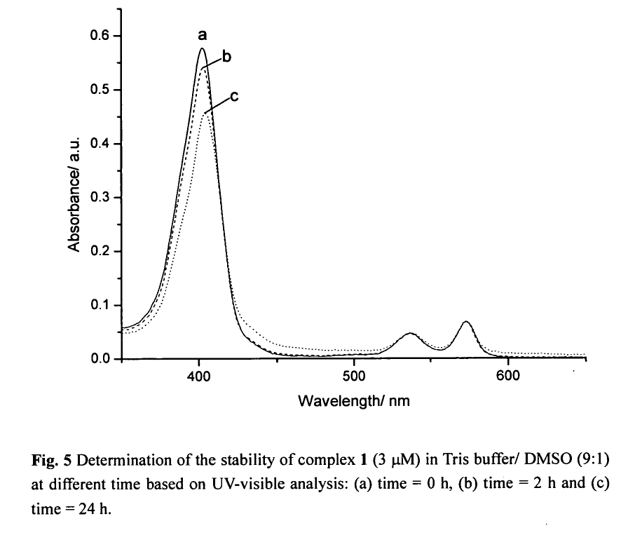

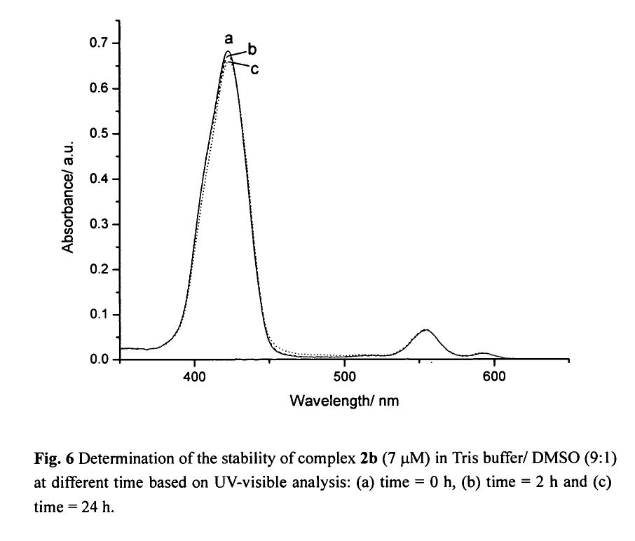

UV-visible experiments. The absorption spectra of 3 μM of complex 1 in DMSO/Tris buffer solution (1:9) were monitored for 24 h. A solution of DMSO/Tris buffer solution (1:9) and complex 1 reveals an intense Soret absorption band at λₘₐₓ=402 nm and two weaker Q bands at λₘₐₓ=536 nm, λₘₐₓ=573 nm. The complex was monitored over 24 h at room temperature and about 23% decrease in the Soret band intensity was recorded without any significant spectral shift in both Soret and Q-bands (FIG. 5). The absorption spectra of 7 μM of complex 2b in DMSO/Tris buffer solution (1:9) were also monitored for 24 h. A solution of DMSO/Tris buffer solution (1:9) and complex 2b reveals an intense Soret absorption band at λₘₐₓ=423 nm and two weaker Q bands at λₘₐₓ=554 nm, λₘₐₓ=592 nm. After 24 h at room temperature, the Soret band intensity of complex 2b was found to decrease in 5% without any significant spectral shift in both Soret and Q-bands (FIG. 6).

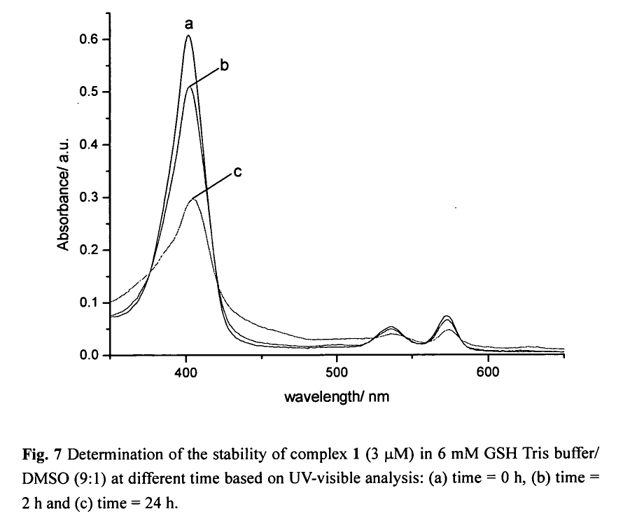

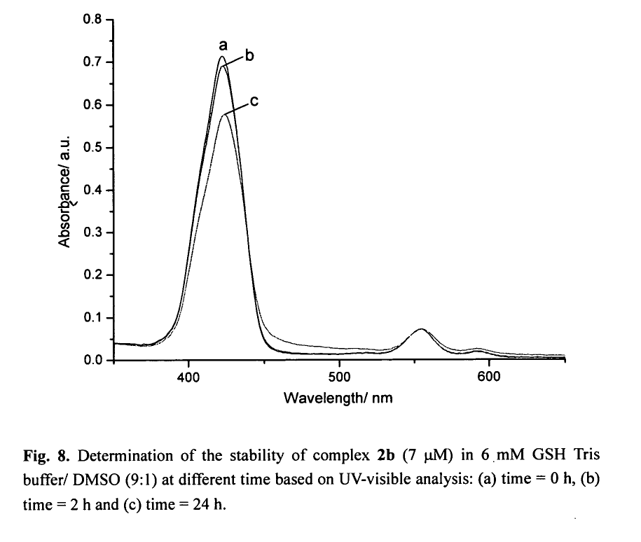

However, in the presence of reduced glutathione [6 mM in DMSO/Tris buffer solution (1:9)], the Soret band intensity of complex 1 was found to decrease 7% and 52% after 2 h and 24 h, respectively. Moreover, concomitant band broadening in spectrum of complex 1 was observed after 24 h. No significant spectral shift and formation of new absorption band were observed. Examination of the Q band region did not reveal any absorption peaks corresponding to the presence of free base porphyrins, and thus demetallization was excluded (FIG. 7). Comparing with the spectra of complex 1, the decrease in Soret band intensity of complex 2b is less significant. The intensities are only found to decrease in 2% and 19% after 2 h and 24 h, respectively. Also, there is no significant spectral shift and formation of new absorption band were observed (FIG. 8).

The Soret band broadening as mentioned before should not be due to demetallization of complexes, since the spectrum can be recovered with addition of minute amount of acetone. Without being limited, probable reasons for the above phenomenon are that aggregation or precipitation of complexes in aqueous buffer occurred, which led to the lowering and broadening in absorption peaks. The addition of acetone helped the complexes to redissolve in buffer so that the absorption peaks can be recovered.

### Example 3

### Cytotoxicity Studies of Lanthanide Porphyrin Complexes Toward Different Cancer and Normal Cell Lines

Examples 3 describe the results of cytotoxicity studies of lanthanide (III) porphyrin complexes 1, 2a and 2b.

As illustrated in Table 3a and 3b, complex 1 showed promising cytotoxic activity toward different cancer cell lines. It was worth noting that complex 1 displayed distinguish cytotoxic effect toward different human liver carcinoma cells, IC₅₀=0.06-0.78 μM, with treatment for 48 h. The IC₅₀ of complex 1 toward the QGY-7703 human liver cancer cell line was 0.06 μM and its multi-drug resistant (QGY-TR50) was 0.54 μM, suggesting that complex 1 may circumvent multi-drug resistant disease. Compared with cisplatin, complex 1 was 760 more effective in eradicating the QGY-7703 human liver cancer cell line (Table 4). Potent cytotoxicity was noted for complex 1, with IC₅₀=0.55 μM toward MCF-7, and was also 60 times higher than that of cisplatin. We found this compound exhibited similar cytotoxicities toward human nasopharyngeal carcinoma cells (SUNE1) and its cisplatin-resistant variant (CNE2) with IC₅₀=2.45 μM and IC₅₀=3.35 μM, respectively (Table 3a).

Cytotoxicities of complexes 2a and 2b toward different cancer cell lines were also examined (IC₅₀=2 μM to 17 μM). They showed less cytotoxic effect when compare with complex 1 (Table 3a and 3b).

TABLE 3a
In vitro growth inhibition of different cancer cell lines by lanthanide
porphyrin complexes.

| | IC₅₀ (μM)^{a} | | | | | normal |
|---|---|---|---|---|---|---|
| | HeLa | HCT-8 | SUNE1 | CNE2 | MCF-7 | CCD-19Lu |
| 1^{b} | 0.47 ± 0.3 | 2.27 ± 0.6 | 2.45 ± 0.2 | 3.35 ± 1.0 | 0.55 ± 0.1 | 5.4 ± 6.8 |
| 2a^{c} | 6.9 ± 0.1 | 15.2 ± 6.1 | 16.2 ± 1.6 | ND | >100 | 25.1 ± 2.6 |
| 2b^{c} | 2.1 ± 0.3 | 5.6 ± 1.5 | 9.4 ± 0.3 | 16.8 ± 0.01 | >100 | 16.0 ± 2.9 |
| Cisplatin^{c} | 14.2 ± 8.2 | 26.1 ± 2.1 | 11.8 ± 1.2 | 57.6 ± 4.6 | 35.4 ± 5.0 | 90.6 ± 42.3 |

TABLE 3b
In vitro growth inhibition of different cancer cell lines by
lanthanide porphyrin complexes.

| | IC₅₀ (μM)^{a} | | | |
|---|---|---|---|---|
| | HepG2 | QGY-TR50 | QGY-7703 | Hep3B |
| 1^{b} | 0.21 ± 0.01 | 0.54 ± 0.14 | 0.06 ± 0.01 | 0.78 ± 0.06 |
| 2a^{c} | 17.1 ± 1.3 | ND | ND | ND |
| 2b^{c} | ND | ND | ND | ND |
| Cisplatin^{c} | 4.76 ± 0.70 | 32.57 ± 1.94 | 45.77 ± 4.13 | 39.75 ± 2.38 |

^{a}Cell viability was measured using MTT assay after incubation with compound at 37° C. for 48 h.
^{b}Compound was dissolved in DMSO:ethanol (1:1) and mixed with respective culture media with final DMSO and ethanol concentration ≦1%.
^{c}Compound was dissolved in DMSO and mixed with respective culture media with final DMSO concentration ≦1%.
^{d}Data was mean ± SD of three independent experiments. ND means not determined.

TABLE 4
Comparison of cytotoxic effect of lanthanide porphyrin complexes with
cisplatin

| | | | QGY- | QGY- | | | | | CCD- |
|---|---|---|---|---|---|---|---|---|---|
| IC₅₀ | HeLa | HepG2 | TR50 | 7703 | HCT-8 | SUNE1 | CNE2 | MCF-7 | 19Lu |
| cisplatin/1 | 30.2 | 22.7 | 60.3 | 763 | 11.6 | 4.8 | 17.2 | 63.89 | 16.8 |
| cisplatin/ | 2.1 | 0.3 | ND | ND | 1.7 | 0.7 | ND | / | 3.6 |
| 2a | | | | | | | | | |
| cisplatin/ | 7.0 | ND | ND | ND | 4.6 | 1.2 | 3.4 | / | 5.6 |
| 2b | | | | | | | | | |

TABLE 5
Comparison of the cytotoxicity of lanthanide porphyrin
complexes toward human hepatocellular carcinoma
cells (QGY-7703) and its multi-drug resistant variant
(QGY-TR50), human nasopharyngeal carcinoma cells
(SUNE1) and its cisplatin-resistant variant (CNE2),
normal lung fibroblast cells (CCD-19Lu) and human
cervical epitheloid carcinoma cells.

| | QGY-TR50/ | CNE2/ | CCD-19Lu/QGY | CCD-19Lu/ |
|---|---|---|---|---|
| IC₅₀ | QGY-7703 | SUNE1 | 7703 | HeLa |
| cisplatin | 0.7 | 4.9 | 2.0 | 11.6 |
| 1 | 7.8 | 1.4 | 83.1 | 9.0 |
| 2a | / | / | / | 7.8 |
| 2b | / | 1.8 | / | 3.6 |

### Example 4

### Interaction Study of Lanthanide Porphyrin Complexes with DNA

Example 4 describes the result of studies showing the interaction of illustrative lanthanide porphyrin complexes 1 and 2b with calf thymus DNA.

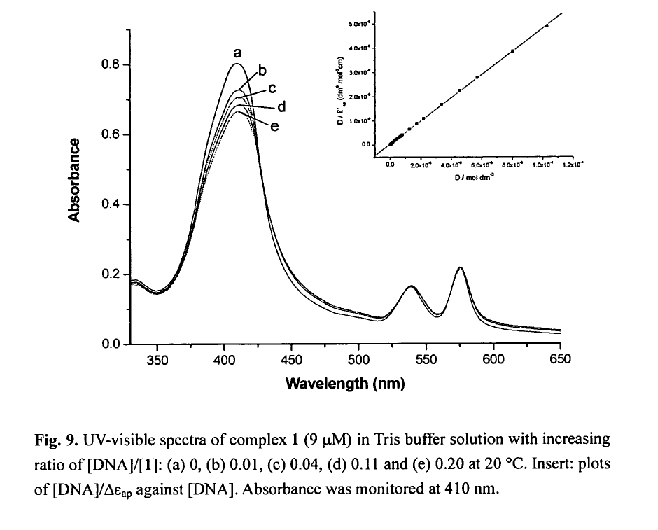

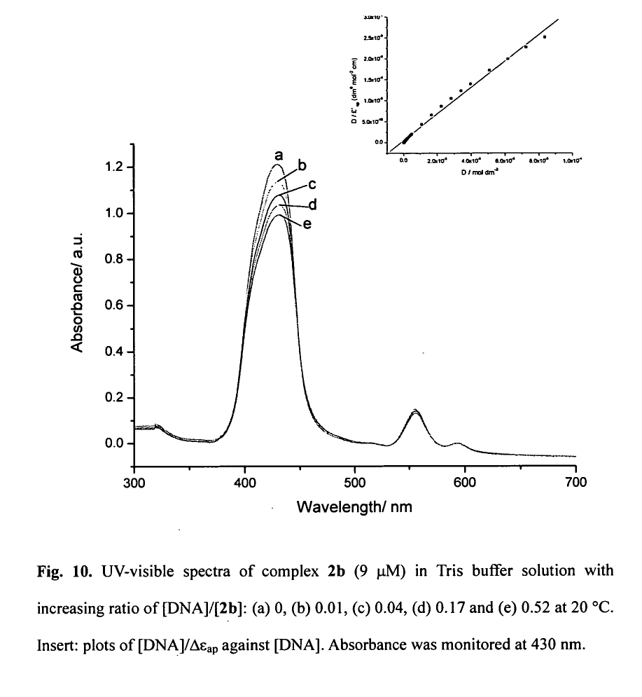

Absorption Titration. Studying the interaction between drugs and DNA is imperative to pharmaceutical chemistry. Since complexation between a ligand molecule and a nucleic acid leads to optical changes, its binding can be studied using spectroscopic methods like fluorescence and electronic absorption spectroscopy (C. V. Kumar, E. H. Asuncion, J. Am. Chem. Soc. 1993, 115, 8547-8553). The binding interactions of lanthanide (III) porphyrin complexes with calf thymus DNA were investigated using UV-vis spectroscopy and depicted in FIGS. 9 and 10 (selected absorption curves were shown for clear plot). By monitoring wavelengths at 410 nm (complex 1) and 430 nm (complex 2b), successive additions of calf thymus DNA to the respective complexes in Tris buffer solution containing <5% DMSO were done until saturation. Moderate hyprochromicities of 27% and 23% in complexes 1 and 2b were found without significant bathochromic spectral shift. Clear isosbestic points at 428 nm and 568 nm for complexes 1 and 488 nm and 528 nm for complex 2b were displayed in each experiment. Notably, strong interactions between complexes 1 and 2b with calf thymus DNA were found, binding constants K=(1.59±0.3)×10⁶ mol⁻¹dm³ and (5.49±0.8)×10⁵ mol¹dm³, respectively. The values of binding constant K were calculated from a plot of D/Δεₐₚ versus D based on the following equation:

D/Δεap=D/Δε+1/(Δε·K)

Where D is the concentration of DNA, Δεₐₚ=|Δε_{A}−Δε_{B}| wherein ε_{A}=A_{obs}/[complex], and Δε=|Δε_{B}−Δε_{F}|; ε_{B} and ε_{F} correspond to the extinction coefficients of DNA-bound lanthanide(III) porphyrin complex adduct and its free complex, respectively. By monitoring absorbance at 410 nm (complex 1) and 430 nm (complex 2b), plots of D/Δεₐₚ against D are inserted in FIGS. 9 and 10. Linear curves in two graphs were plotted with slopes of 4.63×10⁻⁵ (complex 1) and 3.60×10⁻⁵ (complex 2b) (both R=0.99).

### Example 5

### Studies of Apoptosis Induced by Lanthanide(III) Porphyrin Complexes

Example 5 describes the study of cell cycle and programmed cell death of cancer cells treated with the lanthanide porphyrin complexes.

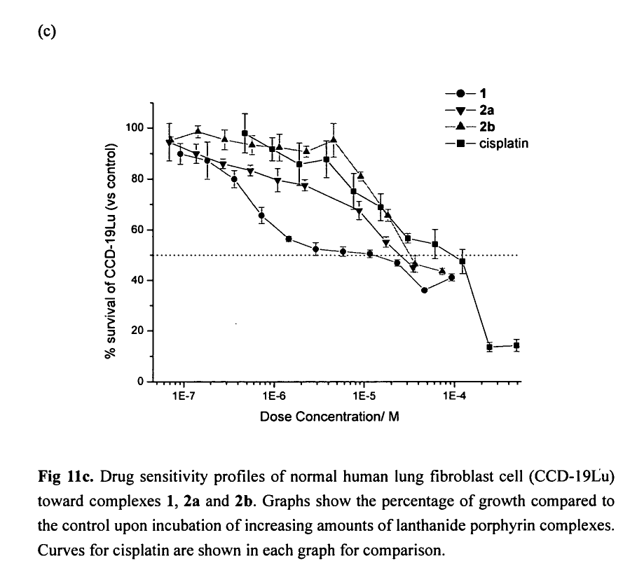

Confocal Microscopic Experiment. By examining cell morphology under scanning confocal microscopy, the type of cell death can be distinguished. Early phase of apoptosis is characterized by the formation of apoptotic bodies when the integrity of the cell membrane is maintained without cell lysis. On the other hand, necrosis is characterized by cell lyses with the leaking out of internal cellular components such as DNA. With ethidium bromide (DNA stain) and acridine orange (cell membrane stain) as staining agents, normal, apoptotic and necrotic cells can be distinguished under laser confocal microscopy (Chan et. al., Inorg. Chem. 2002, 41, 3161-3171). In this experiment, HeLa cells were treated with complex 1 (0.2 μM) for 20 h and complex 2b (10 μM) for 16 h, both living and apoptotic cells were observed. Apoptotic bodies marked by a circle can be found in these cells as shown in FIG. 12.

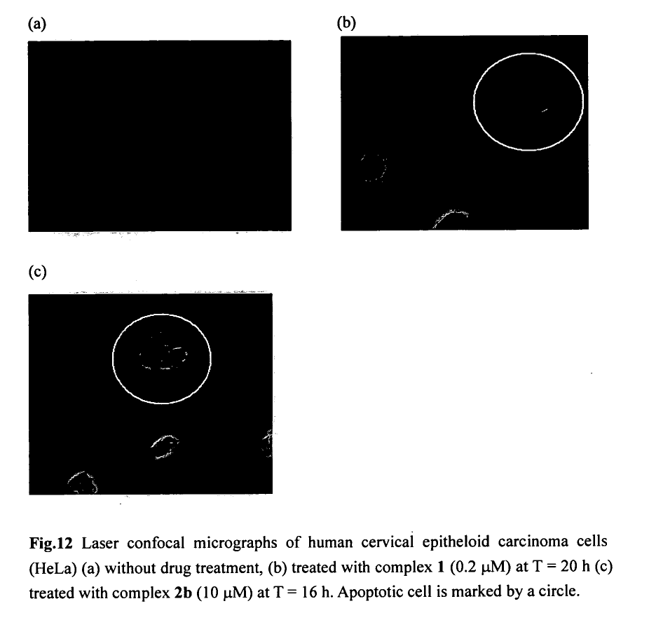

Fluorescence-Activated Cell Sortings (FACS). Cells in different phases of the cell cycle can be differentiated by the DNA content, which can be distinguished by FACS after staining the cells with propidium iodide (PI). Resting cells are said to be in the G₀ phase of the cell cycle. Once the cell is instructed to divide, it enters the active phase of the cell cycle, which is consisted of G₁, S, G₂ and M phase. Treatment of cancer cells with cytotoxic agents may induce cell cycle arrest at a particular phase of the cell cycle. Flow cytometry with PI staining was performed in HeLa cells treated with complex 1 (2 μM) for 0, 6 h, 12 h, 18 h, 24 h, 36 h and 48 h. FIG. 13 illustrates the proportion of cells in different phases of the cell cycle and the percentage of apoptosis at different time points. We noticed that there is no observable change in proportion of cells in G₀-G₁, G2-M and S phase of the cell cycle after the treatment with complex 1. Nevertheless, more than 60% of cells showed DNA fragmentation after treatment with complex 1 for 48 h. Taken together, these data suggested that treatment of HeLa cells with complex 1 induces cell death. Further experiments are essential to investigate the cytotoxic mechanism induced by the lanthanide complex.

Western Blotting Analysis. Apoptotic cell death is mediated by caspases and their regulatory components such as activators and repressors. Treatment of cancer cells with chemotherapeutics usually triggers the activation of a caspase cascade and the cleavage of caspase substrates. Caspase 3, a downstream effector caspase, is one of the main executioners of apoptosis. It plays the role in the proteolytic cleavage of many key proteins such as the nuclear enzyme poly (ADPribose) polymerase (PARP). PARP is an enzyme involved in DNA repair. It is cleaved during apoptosis to a 24 kDa and a 85 kDa fragment representing the N-terminal DNA-binding domain and the C-terminal catalytic subunit, respectively. On the other hand, protein of the BcI-2 family significantly affects the capability of cells to undergo apoptosis by monitoring mitochondrial permeability and the release of cytochrome c. Proapoptotic proteins in this family include Bax, Bak, Bid, Bad, Bok and Bik and anti-apoptotic members include Bcl-2, Bcl-xL, Mcl-1, A1 and Boo (G. Nunez, M. A. Benedict, Y. Hu, N. Inohara, Oncogene 1998, 17, 3237-3245) (D. R. Green, Cell 1998, 94, 695-698).

Cells were washed in cold phosphate-buffered saline (PBS) at 4° C. and lysed in a buffer containing 0.05 M Tis.HCl (pH 7.5), 0.25 M NaCl, 0.005 M EDTA, 0.05 M NaF, 1 mM DTT and 1% Triton. Cell lysates were collected and the protein concentration of cell lysates was measured using a Protein detection kit (Bio-Rad). Equal amount of proteins was resolved in a 12% SDS-PAGE gel and transferred onto nitrocellulose membranes. The membrane was blocked with a blocking solution (1×TBS, 0.1% Tween-20 with 5% non-fat dry milk) at room temperature for 1 h, and washed three times with washing buffer (1×TBS, 0.1% Tween-20) for 5 min each. The membrane was then incubated with primary antibody (Caspase 3, Bid, PARP and β-Actin respectively) for overnight at 4° C. The membrane was washed three times with washing buffer for 5 min each before incubation with the corresponding secondary antibody (horse anti-mouse IgG or goat anti-rabbit IgG). Membrane was washed again and signals were detected using an ECL detection kit (Amersham Pharmacia Biotech) followed by the exposure to X-ray films (Fujifilm).

Preparation of Mitochondrial and Cytosolic Fractions. Subcellular fractionations of cells were carried out as described previously with modifications (ref). In brief, cells were harvested by centrifugation at 1000 g for 5 min at 4° C. Cell pellets were washed once with ice-cold phosphate-buffered saline and resuspended in 5 volumes of buffer A (20 mM HEPES-KOH, pH 7.5, 10 mM KCl, 1.5 mM MgCl₂, 1 mM sodium EDTA, 1 mM sodium EGTA, 1 mM dithiothreitol, 0.1 mM phenylmethylsulfonyl fluoride, 1× proteinase inhibitor mix) containing 250 mM sucrose. After incubation on ice for 15 min, the cells were homogenized with a 25-gauge needle for 20 strokes, and the homogenates were centrifuged at 1,000 g for 5 min at 4° C. The supernatant was collected and centrifuged at 10,000 g for 15 min at 4° C., and the mitochondria-enriched pellets were resuspended in buffer A. The supernatants of the 10,000 g spin were further centrifuged at 60,000 g for 1 h at 4° C., and the supernatants were designated as the cytosolic fraction. The protein concentration of cell lysates in different portions was measured using a protein detection kit (Bio-Rad). Equal amount of proteins was loaded on a 12% SDS-PAGE gel and transferred onto nitrocellulose membranes. The membrane was blocked with a blocking solution (1×TBS, 0.1% Tween-20 with 5% non-fat dry milk) at room temperature for 1 h, and washed three times with washing buffer (1×TBS, 0.1% Tween-20) for 5 min each. The membrane was then incubated with cytochrome c antibody for overnight at 4° C. The membrane was washed three times with washing buffer for 5 min each before incubation with the goat-anti-rabbit IgG as secondary antibody. Signals were detected using an ECL detection kit (Amersham Pharmacia Biotech) followed by exposure to X-ray films (Fujifilm).

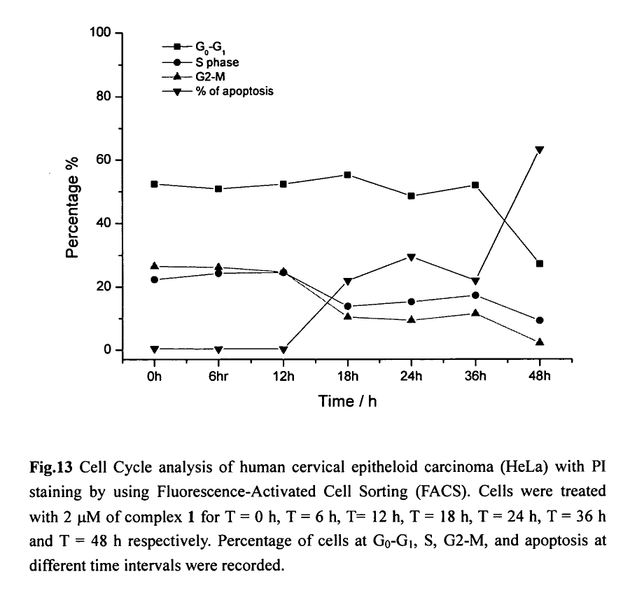

After treatment with complex 1 for different time intervals, the expressions of caspase 3, Bid and PARP were detected by the Western blotting analysis. Our data shows that the expressions of caspase 3 and Bid decrease dramatically in HeLa cells treated with complex 1 (2 μM) for 32 h. On the other hand, there is a gradual increase in the level of cleaved PARP (85 kDa) after the treatment (FIG. 14a). Furthermore, there is an increase in the amount of cytochrome c found in the cytosolic fraction, whereas a corresponding decrease is found in the mitochondria (FIG. 14b). Moreover, treatment with complex 1 leads to a reduction in the level of un-cleaved Bid. Taken together, our data suggested that complex 1 treatment leads to apoptosis in HeLa cells.

### Example 6

In Vivo Studies of Antitumor Activity of Lanthanide Porphyrin Complex in the Murine Xenograft Model Using the Multi-Drug Resistant QGY-TR50 Hepatocellular Carcinoma Cells

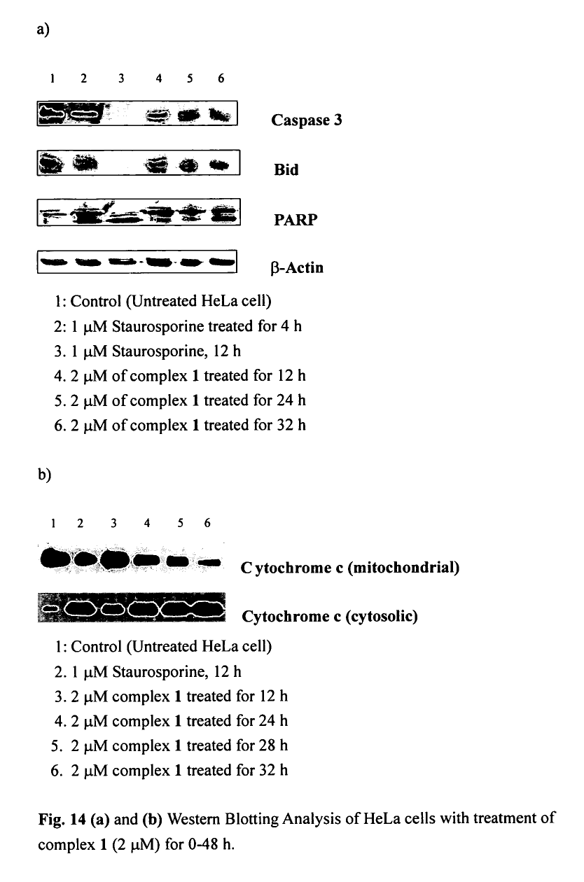

The anti-tumor effect of complex 1 was examined in the xenograft mice model. A complex 1 suspension was prepared by dissolving the complex in 1% EtOH and mixed with a solution containing 0.2% sodium carboxymethyl cellulose (Sigma) and 0.1% Tween 80 (Sigma) in PBS. A dose tolerance test was conducted to determine the appropriate dose ranges for complex 1. Five- to seven-week-old male nude mice (each group contains 4 mice) were implanted with 2×10⁶ multi-drug resistant liver tumor cells (QGY-TR50) subcutaneously in the mid-dorsal region. Tumors were allowed to grow for 5 days. When the tumors became palpable (3-4 mm diameter), complex 1 (at 10 mg/kg/day in 0.1 ml) was administered via i.p. daily for 22 days. For the control group, mice received identical volumes of the vehicle. The dimensions of the tumors were measured twice weekly with calipers, and tumor volumes were estimated using two-dimensional measurements of length and width and calculated with the formula [l×(w)²]×0.52 (l, length; w, width) according to described procedures (M. Guba, P. von Breitenbuch, M. Steinbauer, G. Koehl, S. Flegel, M. Hornung, C. J. Bruns, C. Zuelke, S. Farkas, M. Anthuber, K. W. Jauch, E. K. Geissler, Nat Med 2002, 8, 128-135). The weights of the mice were also recorded twice weekly throughout the course of treatment. When compared to the control group, treatment with complex 1 at 10 mg/kg/day resulting in a 40% inhibition of the tumor volume (FIG. 15).

### Example 7

### Studies of Autophagic Cell Death Induced by Lanthanide(III) Porphyrin Complexes

Example 7 describes the study of autophagic cancer cell death induced by the lanthanide porphyrin complexes.

The GFP-LC3 plasmid was generously provided by Prof. Tamotsu Yoshimori (National Institute of Genetics, Shizuoka, Japan). Photographs of cells were taken with laser confocal microscope (Zeiss Axiovert 100M) and 208S Philips transmission electron microscopy.

Transmission Electron Microscopy. HeLa cells (12×10⁴) were seeded on 60 mm plate. Cells were treated with 1 (1 μM) for 2 h, respectively. Cells were then trypsinized, washed twice with PBS and fixed with ice-cold glutaraldehyde (2.5% in 0.1 M cacodylate buffer, pH 7.4) for 30 min. After primary fixation, cells were washed with cacodylate buffer, post-fixed with 1% osmium tetraoxide for 30 min. Osmium tetraoxide was removed and the cell pellet was washed with cacodylate buffer. Pre-warmed gelatin was added to the cell pellet and immediately centrifuged at 2500 rpm for 5 min. The gelatin was dehydrated by immersing in 30% EtOH, 50% EtOH, 70% EtOH once, and in 90% EtOH twice for 5 min and finally in absolutely EtOH for 10 min for three times. The dehydrated sample was washed with propylene oxide twice for 5 min and then immersed in propylene oxide:resin (1:1) for overnight. The sample was immersed in pure resin for 30 min before embedded in Polybed. Specimens were examined using 208S Philips transmission electron microscopy.

Subcellular Distribution of LC3. HeLa cells were transiently transfected with a plasmid expressing green fluorescent protein-tagged LC3 (GFP-LC3) using GeneJuice (Novagen, USA). After overnight incubation, HeLa cells were treated 1 (1 μM) for 1, 2, 3, and 4 hours respectively. Green fluorescence of GFP-LC3 was examined under the Olympus IX71 microscope (Melville, N.Y.). Images were captured with CCD digital camera RT COLOR, Spot (Diagnostic Instruments, Inc., Melville, N.Y.).

Effect of 3-MA on 1-induced cytotoxicity. Cells were seeded on 96-well microtitre plates (8×10³ cells per well). After overnight incubation, the cells were pre-incubated with 3-methyladenine (3-MA, 10 mM) for 1 hour and then in the presence of 1 (DMSO<1%) for 24 h. Cell viability was measured by MTT assay as described before.

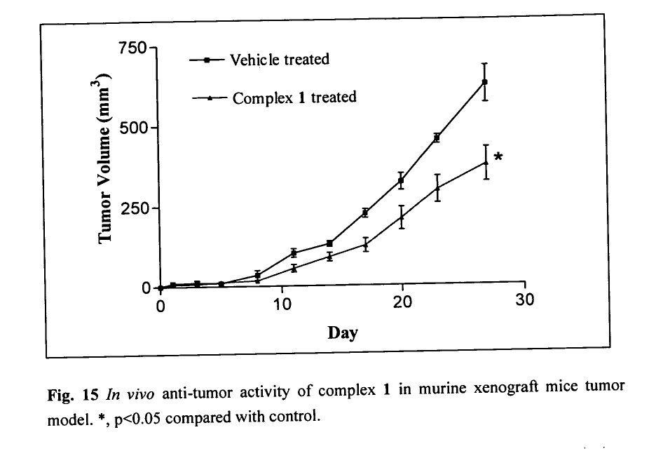

Cells treated with 1 were subjected to transmission electron microscopic analysis. The electron micrographs revealed that vesicular structures reminiscence of autophagic vacuoles (autolysosomes) induced by temozolomide and As₂O₃ was observed at 2 h after treatment with 1, which marks the characteristic vesicular organelle (AVO) formed during autophagy (FIG. 16).

The redistribution of microtubule-associated protein 1 light chain 3 (LC3) was also examined. LC3 is a maker of autophagosomes in mammalian cells which the localization changes from a diffuse cytosolic pattern to a punctuate pattern representing its recruitment to the autophagosomal membrane during the induction of autophagy. HeLa cells were transfected with plasmid encoded a fusion protein of GFP and LC3 (GFP-LC3), so that the cellular trafficking of LC3 can be directly visualized by live fluorescence microscopy. Cells treated with 1 (1 μM) exhibited a time-dependent increase in punctuated green fluorescence representing autophagosomes followed by the appearance of autolysosome.

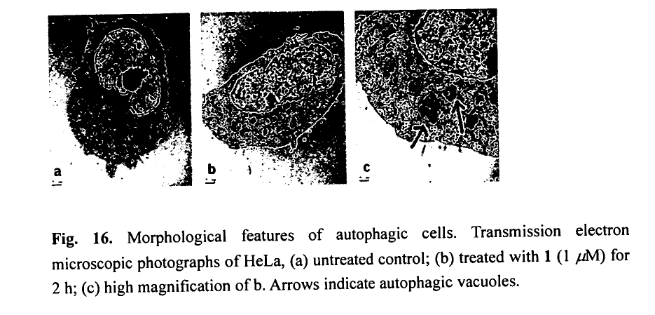

The effect of autophagy inhibitor on the cytotoxicity of the lanthanide complex 1 was also examined. As shown in FIG. 17, pretreatment with 3-methyladenine (3-MA), a known inhibitor of autophagy, significantly increases cell viability of 1-treated cells (P<0.001), whereas pre-treatment with 3-MA alone did not affect cell viability.

What is claimed is:

1. A method for the treatment of cancer comprising administering to a patient in need thereof a pharmaceutical composition containing an effective amount of a lanthanide porphyrin complex and a pharmaceutically acceptable carrier.

2. The method according to claim 1, wherein the lanthanide porphyrin complex induces programmed cell death of cancer cells.

3. The method according to claim 1, wherein programmed cell death is caused by apoptosis.

4. The method according to claim 1, wherein the lanthanide porphyrin complex inhibits tumor growth in the patient.

5. The method of claim 4, wherein the tumor is cervical epitheloid carcinoma, human hepatocellular carcinoma, breast cancer, human coloadenocarcinoma or nasopharyngeal carcinoma.

6. The method according to claim 1, wherein the patient is a mammal.

7. The method according to claim 5, wherein the mammal is a human.

8. The method according to claim 1, wherein the lanthanide is chelated by porphyrin with a pharmaceutically acceptable anion, counter-ion, or a pharmaceutically acceptable carrier and isotonally coordinated with a solvent.

9. The method according to claim 1, wherein the lanthanide portion of the complex is cerium, neodymium, samarium, europium, gadolinium, terbium, dysprosium, holmium, ytterbium or lutetiumion.

10. A method for inhibiting tumor growth in a patient in need thereof, comprising administering to the patient an effective tumor inhibiting amount of a lanthanide porphyrin complex, where in the lanthanide porphyrin complex is represented by structural formula I in a pharmaceutically acceptable vehicle: 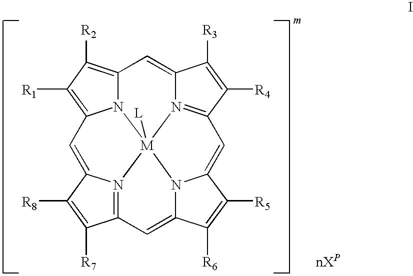
or a pharmaceutically acceptable salt thereof, wherein:
M is a lanthanide metal;
L is any mono-, di-, tri-, tetra- or polydentate ligand; and which L includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s);
R₁-R₈ are each neutral or negatively charged moiety, and are each independently hydrogen, alkyl, substituted alkyl, alkenyl, substituted alkenyl, alkynyl, substituted alkynyl, phenyl, substituted phenyl, halo, nitro, hydroxyl, alkoxyl, substituted alkoxyl, phenoxyl, substituted phenoxyl, aroxyl, substituted aroxyl, alkylthio, substituted alkylthio, phenylthio, substituted phenylthio, phenylthio, substituted phenylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, substituted phosphoramide, C₁-C₂₀ cyclic, substituted C₁-C₂₀ cyclic, heterocyclic, substituted heterocyclic, amino acid, peptide, or polypeptide group;
each X^{P} is independently a pharmaceutically acceptable counter-ion;
m is an integer ranging from −4 to 1;
P is an integer ranging from −3 to 3; and
n is equal to the absolute value of m/p.

10. The method of claim 9, wherein R₁, R₂, R₃, R₄, R₅, R₆, R₇ and R₈ are each -ethyl; M is ytterbium, L is hydroxyl.

11. The method of claim 9, wherein R₁, R₄, R₆ and R₈ are each -ethyl; R₂, R₃, R₅ and R₇ are each -methyl; M is ytterbium, L is hydroxyl.

12. The method of claim 9, wherein R₄ and R₆ are each -ethyl; R₂, R₃, R₅ and R₇ are each -methyl; R₁ and R₈ are each -methyl-3-propanoate; M is ytterbium, L is hydroxyl.

13. The method of claim 9, wherein R₄ and R₆ are each -ethyl; R₂, R₃, R₅ and R₇ are each -methyl; R₁ and R₈ are each -methyl-3-propanoic acid; M is ytterbium, L is hydroxyl.

14. The method of claim 9, wherein R₄ and R₆ are each —CHOHCH₃; R₂, R₃, R₅ and R₇ are each -methyl; R₁ and R₈ are each -methyl-3-propanoate; M is ytterbium, L is hydroxyl.

15. The method of claim 9, wherein R₄ and R₆ are each —CHOHCH₃; R₂, R₃, R₅ and R₇ are each -methyl; R₁ and R₈ are each -methyl-3-propanoic acid; M is ytterbium, L is hydroxyl.

16. The method of claim 9, wherein R₄ and R₆ are each —CH₂═CH₂; R₂, R₃, R₅ and R₇ are each -methyl; R₁ and R₈ are each -methyl-3-propanoate; M is ytterbium, L is hydroxyl.

17. The method of claim 9, wherein R₄ and R₆ are each —CH₂═CH₂, R₂, R₃, R₅ and R₇ are each -methyl; R₁ and R₈ are each -methyl-3-propanoic acid; M is ytterbium, L is hydroxyl.

18. The method according to claim 8, wherein the lanthanide porphyrin is monomeric, dimeric or polymeric form.

19. A method for treatment of a cancer comprising administering to a patient in need thereof a pharmaceutical composition comprising an effective amount of a lanthanide porphyrin complex in a pharmaceutically acceptable vehicle wherein the lanthanide complex is represented by structural formula II: 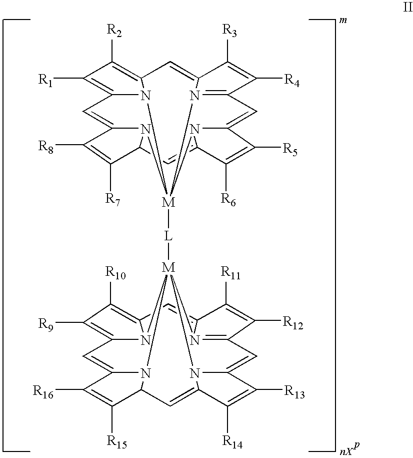
or a pharmaceutically acceptable salt thereof, wherein:
M is a lanthanide metal;
L is any mono-, di-, tri-, tetra- or polydentate ligand; and which L includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s);
R₁-R₈ are each neutral or negatively charged moiety, and are each independently hydrogen, alkyl, substituted alkyl, alkenyl, substituted alkenyl, alkynyl, substituted alkynyl, phenyl, substituted phenyl, halo, nitro, hydroxyl, alkoxyl, substituted alkoxyl, phenoxyl, substituted phenoxyl, aroxyl, substituted aroxyl, alkylthio, substituted alkylthio, phenylthio, substituted phenylthio, phenylthio, substituted phenylthio, cyano, isocyano, substituted isocyano, carbonyl, substituted carbonyl, carboxyl, substituted carboxyl, amino, substituted amino, amido, substituted amido, sulfinyl, substituted sulfinyl, sulfonyl, substituted sulfonyl, sulfonic acid, substituted sulfonic acid, phosphonato, substituted phosphonato, phosphoramide, substituted phosphoramide, C₁-C₂₀ cyclic, substituted C₁-C₂₀ cyclic, heterocyclic, substituted heterocyclic, amino acid, peptide, or polypeptide group;
each X^{P} is independently a pharmaceutically acceptable counter-ion;
m is an integer ranging from −4 to 1;
P is an integer ranging from −3 to 3;
n is equal to the absolute value of m/p; and
a pharmaceutically acceptable carrier.

20. The method of claim 19, wherein R₁, R₂, R₃, R₄, R₅, R₆, R₇, R₈, R₉, R₁₀, R₁₁, R₁₂, R₁₃, R₁₄ R₁₅ and R₁₆ are each -ethyl; M is ytterbium, L is two hydroxyls.

21. The method of claim 19, wherein R₁, R₂, R₃, R₄, R₅, R₆, R₇, R₈, R₉, R₁₀, R₁₁, R₁₂, R₁₃, R₁₄ R₁₅ and R₁₆ are each -ethyl; M is ytterbium, L is an oxalate.

22. A method for treating cancer in a patient suffering therefrom, comprising administering an effective amount of a lanthanide porphyrin is represented by structural formula III in a pharmaceutically acceptable vehicle: 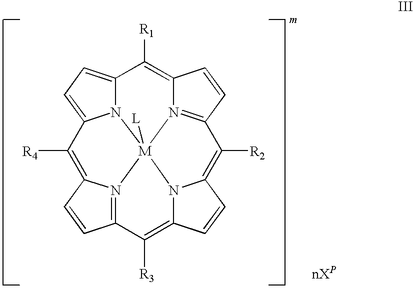
or a pharmaceutically acceptable salt thereof, wherein:
M is a lanthanide metal;
L is any mono-, di-, tri-, tetra- or polydentate ligand; and which L includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s);
R₁-R₈ are each neutral or negatively charged moiety, and are each independently phenyl, halo substituted phenyl, alkyl substituted phenyl, alkenyl substituted phenyl, alkynyl substituted phenyl, aryl substituted phenyl, cyano substituted phenyl, isocyano substituted phenyl, sulfinyl substituted phenyl, sulfonyl substituted phenyl, sulfonic acid phosphonato substituted phenyl, phosphoramide substituted phenyl, polyaryl substituted phenyl, C₁-C₂₀ cyclic substituted phenyl, amino acid substituted phenyl, peptide substituted phenyl or polypeptide substituted phenyl;
each X^{P} is independently a pharmaceutically acceptable counter-ion;
m is an integer ranging from −4 to 1;
P is an integer ranging from −3 to 3; and
n is equal to the absolute value of m/p.

23. The method according to claim 22, wherein the lanthanide porphyrin can exist in monomeric, dimeric or polymeric form.

24. A method for treating cancer in a patient, comprising administering to the patient an effective amount of a lanthanide porphyrin complex represented by structural formula IV in a pharmaceutically acceptable vehicle: 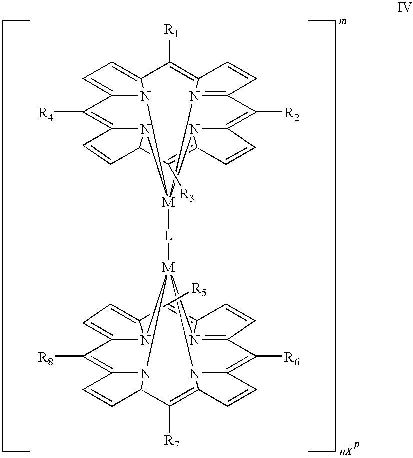
or a pharmaceutically acceptable salt thereof, wherein:
M is a lanthanide metal;
L is any mono-, di-, tri-, tetra- or polydentate ligand; and which L includes but not limited to one or more than one coordinating solvent molecule(s), halide(s), oxo(s), cyanide(s), cyanate(s), thiocyanate(s), isocyanate(s), isothiocyanate(s), hydroxyl(s), alkoxyl(s), heterocyclic(s), acetylacetonate(s), acetate(s), trifluoromethanesulfonate(s), imidazole(s) or oxalate(s);
R₁-R₈ are each neutral or negatively charged moiety, and are each independently phenyl, halo substituted phenyl, alkyl substituted phenyl, alkenyl substituted phenyl, alkynyl substituted phenyl, aryl substituted phenyl, cyano substituted phenyl, isocyano substituted phenyl, sulfinyl substituted phenyl, sulfonyl substituted phenyl, sulfonic acid phosphonato substituted phenyl, phosphoramide substituted phenyl, polyaryl substituted phenyl, C₁-C₂₀ cyclic substituted phenyl, amino acid substituted phenyl, peptide substituted phenyl or polypeptide substituted phenyl;
each X^{P} is independently a pharmaceutically acceptable counter-ion;
m is an integer ranging from −4 to 1;
P is an integer ranging from −3 to 3; and
n is equal to the absolute value of m/p.

25. The method of claim 24, wherein R₁, R₂, R₃, R₄, R₅, R₆, R₇ and R₈ are each -phenyl; M is ytterbium, L is two hydroxyls.

26. The method of claim 24, wherein R₁, R₂, R₃, R₄, R₅, R₆, R₇ and R₈ are each -phenyl; M is ytterbium, L is an oxalate.
